(19) United States
(12) Patent Application Publication
(10) Pub. No.: US 2013/0283403 A1
(43) Pub. Date: Oct. 24, 2013

(54) ZEBRAFISH SEIZURE MODEL, METHOD FOR ESTABLISHING THE SAME, AND METHOD FOR SCREENING ANTIEPILEPTIC DRUG USING THE SAME

(71) Applicant: NATIONAL CHENG KUNG UNIVERSITY, Tainan City (TW)

(72) Inventors: Tzu-Fun FU, Tainan City (TW); Gang-Hui LEE, Tainan City (TW)

(73) Assignee: National Cheng Kung University, Tainan City (TW)

(21) Appl. No.: 13/867,191

(22) Filed: Apr. 22, 2013

(30) Foreign Application Priority Data
Apr. 20, 2012 (TW) 101114169

(51) Int. Cl. A61K 49/00

(52) U.S. Cl. CPC A61K 49/0008
USPC 800/3; 800/9

(57) ABSTRACT

A zebrafish seizure model, a method for establishing the same, and a method for screening for antiepileptic drug using the same are disclosed. The method for establishing the zebrafish seizure model comprises the following steps: placing a zebrafish in a medium containing an inducing compound represented by the following formula (I) to induce seizure-like symptom in zebrafish:

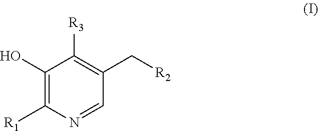

wherein, the definitions of R₁, R₂, R₃ are the same as those defined in the specification.

### BACKGROUND OF THE INVENTION

1. Field of the Invention

The present invention relates to a zebrafish seizure model, a method for establishing the same, and a method for screening for antiepileptic during the same. More specifically, the present invention relates a method for establishing a zebrafish seizure model using ginkgotoxin, and a method for screening for antiepileptic using the same.

2. Description of Related Art

Epilepsy/seizure is a common neurological disorder affecting more than 50 million people worldwide and could be induced in people of all ages. Further, epilepsy cannot be cured completely once it occurs in a person.

The cause of seizure is mostly due to abnormal neuron activity in the cerebral hemisphere, which disables proper functioning of person's brain, and therefore decapacitates sensory perception and mental processing, so diminishing motor skill or leading to epileptic convulsion. In a worsen state, this transient syndrome can present a threat to a patient's life and safety.

On the prevention front against seizure, the current approach for treating seizure of a minor degree is controlled by drugs mediation. For situations beyond help by pills (for example, intensive seizure condition), surgical operation becomes the obvious choice. Notwithstanding option for surgical operation, seizure patients will be burdened with persistent condition of drug taking for life in order for effective prevention of seizure recurrence. Therefore, many antiepileptic drugs have been developed at present, such as carbamazepine, depakene, phenyloin, trileptal, zonegran which all show excellent curative effects. However, over 30% of patients have symptoms which still could not be managed by prevailing drugs, which is not discounting the possibility that these drugs may induce collateral actions.

The etiology and pathogenesis of the seizures are very complicated, and not all presently available antiepileptic drugs are appropriate for every patient. Consequently, it is an immediate issue in view of studies of drugs relative to antiepileptic.

### SUMMARY OF THE INVENTION

The objective of the present invention is to provide a method for establishing a zebrafish seizure model or convulsions model in an expedient and less costly manner.

The other objective of the present invention is to provide a zebrafish seizure model as a simulated means for screening for antiepileptic or anti-convulsion drugs.

A further objective of the present invention is to provide a method for screening for antiepileptic drugs using the zebrafish seizure model of the present invention, which is capable of establishing a screening plat to quickly screen for antiepileptic drugs or anti-convulsions drugs.

To achieve the objective, the method for establishing the zebrafish seizure model of the present invention includes the following steps: placing a zebrafish in a medium containing an inducing compound represented by the following formula (I) to induce seizure-like symptom in zebrafish:

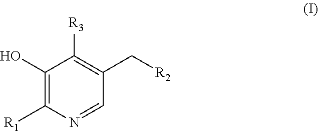

wherein, R₁ is a alkyl group having 1 to 6 carbon atoms, R₂ is a hydrogen atom, a hydroxyl group (—OH), a —O—P(═O)(OH)₂ or a alkyl group having 1 to 6 carbon atoms, and R₃ is a alkyl group having 1 to 6 carbon atoms, an alkoxy group having 1 to 6 carbon atoms, or a ether group having 1 to 6 carbon atoms.

Besides, the present invention further provides a zebrafish seizure model which is established through the above-mentioned method for establishing the zebrafish seizure model.

Even more, the present invention also provides a method for screening for antiepileptic drugs comprising the following steps: (A) providing a zebrafish seizure model, which is established through the above-mentioned method for establishing the zebrafish seizure model; and (B) adding a drug candidate to a culture medium and observing the convulsion state of the zebrafish. A toned-down zebrafish convulsion state is indicative of a screened drug having antiepileptic efficacy.

In the present invention, the zebrafish seizure model is established through using ginkgotoxin analogs, and screening for antiepileptic drugs using the seizure model. Comparing to screening for conventional antiepileptic drugs adopting animal model, the present invention uses the zebrafish to establish the seizure model. The zebrafish animal model is a convenient, cheap and efficient model because the zebrafish has the following benefits to offer: many of zebrafish's organic systems are similar to those of humans', external fertilization and transparent embryo make for more streamlined observation possible, fecundity is fast and productive, induced mutation and related gene experiment can be easily given way to establish, and low expense on culturing is more economically manageable.

At present, there is the zebrafish disease model to induce seizure using pentylenetetrazole (PTZ), but the zebrafish model blocks mainly a combination of inhibiting nervous conductors, gamma-aminobutyric acid (GABA), and thereof in a competitive way to induce seizure. However, the present invention uses ginkgotoxin analogs to induce the zebrafish seizure model, for which the mechanism for inducing seizure is to inhibit synthesis of GABA by inhibiting vitamin B6 activity in order to cause imbalance between excitatory and inhibitory neurotransmitters. Therefore, the used ginkgotoxin analogs can be regarded as a vitamin B6 antagonistic agent, and the pathogenic mechanism that is led on by the analogs are not involved in the competitive inhibition of the GABA receptor. The zebrafish model is induced by using ginkgotoxin analogs which has different specificity and higher sensitivity to drugs, as opposed to the PTZ-induced zebrafish model; therefore, it is advantageous for screening for antiepileptic drugs.

For the zebrafish seizure model, the method for establishing the same, and the method for screening for antiepileptic drugs using the same of the present invention, the zebrafish is preferably a zebrafish embryo. Besides the benefits of time-effectiveness and cost-effectiveness, the therapeutic effect of a drug and its specific toxicity to each organ can be monitored at the same time when a seizure model is established with living zebrafish or zebrafish larva and used for drug screening. Using zebrafish seizure model will allow to avoid the problem commonly encountered while using cells for drug screening. That is the obtained drug will end up be toxic to experimental animal and human, Besides, the effectiveness of drug for relieving seizure symptoms and convulsion cannot be monitored using cultured cells, revealing an absolute requirement for live animal. In addition, the required drug dosage when using zebrafish embryos/larvae for drug screening will be much less than that when using rodents or mammals due to the small size and rapid growth of zebrafish embryos. Meanwhile, the externally development and transparent appearance of zebrafish embryos also facilitate the observation for drug efficacy. Furthermore, most small molecules and compounds can pass directly through larvae and embryonic outer skin. Therefore, the tested drugs can be added to a 96 sieve containing embryos to perform drugs screening, making zebrafish embryo is a highly effective platform for drug screening.

For the zebrafish seizure model, the method for establishing the same, and the method for screening for antiepileptic drugs of the present invention, in the induced compounds represented by formula (I), R₁ is preferably a methyl or ethyl, R₂ is preferably a hydroxyl group, —O—P(═O)(OH)₂, methyl or ethyl, and R₃ is an ether group having 1 to 6 carbon atoms. More preferably, R₂ is a hydroxyl group, and R₃ is —C₁₋₃—O—C₁₋₃. Most preferably, the induced compound is represented by the

following formula (II) or (III):

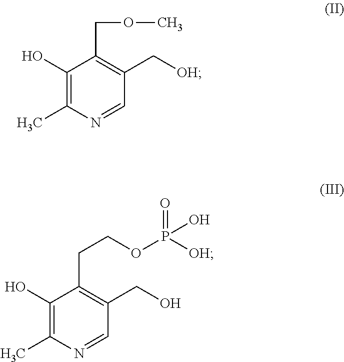

wherein, the compounds represented by formula (III) are phosphates of compounds represented by formula (II).

As the compound represented by formula (II), the chemical name of the compound is 4-O-methylpyridoxine. The compound is a micro-toxin included in the ginkgo seeds which is generally referred to as ginkgotoxin.

For the zebrafish seizure model, a method for establishing the same, and a method for screening for antiepileptic drugs of the present invention, the medium could be a regular medium for incubating the zebrafish or the zebrafish embryo, such as water. Preferably, a concentration for inducing compound in the medium is 0.05 to 1.5 mM; more preferable it is 0.2 to 0.8 mM.

Further, in the method for screening for antiepileptic drugs of the present invention, screened drug candidates are added to the medium, after the zebrafish seizure model is established by using the induced compounds (ginkgotoxin analogs), to perform screening for antiepileptic drugs or the convulsion drugs. Or, the ginkgotoxin analogs and the drugs to be screened can be added simultaneously to the mediums incubating zebrafish, so as to perform antiepileptic drugs or the convulsion drugs screening. Herein, the convulsion state of the zebrafish could be observed by eyes or a microscope; it is more preferable to use optical dissecting microscope.

In addition to the use as a screening means for seizure drug, the zebrafish seizure model and a method for establishing the same of the present invention could be used as an animal model for researching vitamin B6 deficiency, so as to screen for antiepileptic drugs related to vitamin B6 deficiency.

### BRIEF DESCRIPTION OF THE DRAWINGS

FIG. 1A is a figure showing statistical result of the induced zebrafish seizure model after 1 hour according to example 1 of the present invention;

FIG. 1B is a figure showing statistical result of the induced zebrafish seizure model after 2 hours based on example 1 of the present invention;

FIGS. 2A and 2B are figures showing final results of a zebrafish having undergone 2 hours of processing on the 3^{rd} day after fertilization, where the zebrafish is one from example 2 of the current invention;

FIGS. 3A and 3B are figures showing final results of a zebrafish having undergone 2 hours of processing on the fifth day after fertilization, wherein the zebrafish is one from example 2 of the current invention;

FIGS. 4A and 4B are figures showing final results of a zebrafish having undergone 3 days of processing after 6 hours of waiting time after fertilization, where the zebrafish is one from example 2 of the current invention; and

FIGS. 5A and 5B are comparative graphs of the ginkgotoxin and PTZ-induced zebrafish seizure models according to example 3 of the present invention.

### DETAILED DESCRIPTION OF THE PREFERRED EMBODIMENT

Hereafter, examples will be provided to illustrate the embodiments of the present invention. Other advantages and effects of the invention will become more apparent from the disclosure of the present invention. Other various aspects also may be practiced or applied in the invention, and various modifications and variations can be made without departing from the spirit of the invention based on various concepts and applications.

### Materials and Animals

The zebrafish (Dania rerio, AB strain) used in the present invention are obtained from NTHU-NHRI Zebrafish Core Facility in Taiwan, which are bred and maintained at 28° C. of the water temperature in a diurnal cycle (10 hours of light/14 hours of darkness) based on the standard procedure. Green fluorescence transgenic zebrafish embryo Tg (alx: GFP) are obtained from Laboratory of Developmental Gene Regulation/RIKEN Brain Science Institute and National Institutes of Natural Science, Japan. All experiments had followed the Animal Use Protocol (IACUC Approval No: 99059) approved by the Institutional Animal Care and Use committee, National Cheng Kung University.

The ginkgotoxin represented by formula (II) and ginkgotoxin phosphate represented by formula (III) are obtained from Dr. Martin Safo (Institute of Structural Biology and Drug Discovery/Virginia Commonwealth University, Richmond Va.).

### Establishing Zebrafish Seizure Model and Drug Treatment

A solution of the ginkgotoxin is stored below −20° C. The ginkgotoxin concentrate of 40 mM is added to a fish water to keep the concentrations between 0.2 mM and 1 mM while the zebrafish seizure model is induced. The zebrafish is incubated in the fish water without ginkgotoxin for the control group.

Pyridoxal-5′-phosphate and GABA stock solution of 40 mM are used when measuring the response of zebrafish embryos seizure model with respect to effects of ant-convulsant drugs. Anticonvulsant drugs, such as gabapentin, phenyloin, and primidone, are dissolved in DMSO to make 100 mM concentration. During experiments, anticonvulsant drugs and ginkgotoxin are added to the fish water simultaneously, or the anticonvulsant drugs are added to the fish water three hours before the zebrafish behaviors are recorded and analyzed. This will be described in detail in the following.

The swimming behavior of zebrafish were was observed directly by naked eyes to determine the test results, and a quantified standard is made by a swimming distance and velocity of the zebrafish. At least 10 zebrafish were used for each group.

Besides, the zebrafish embryos are placed in 200 μl fish water in a concave on microscope slide. Herein, the zebrafish embryos are observed and recorded under a high-resolution dissecting microscope (Panasonic Digital video camera, DMC-FX55GT). Further, each condition, such as the swimming distance and velocity, of zebrafish are analyzed and quantified by a locomotion tracking system (EthoVision XT8.0 locomotion tracking system® (Noldus Information Technology, Inc., Leesburg, Va., USA)). The zebrafish is incubated in the fish water without ginkgotoxin for the control group.

### Example 1 Establishing the Zebrafish Seizure Model

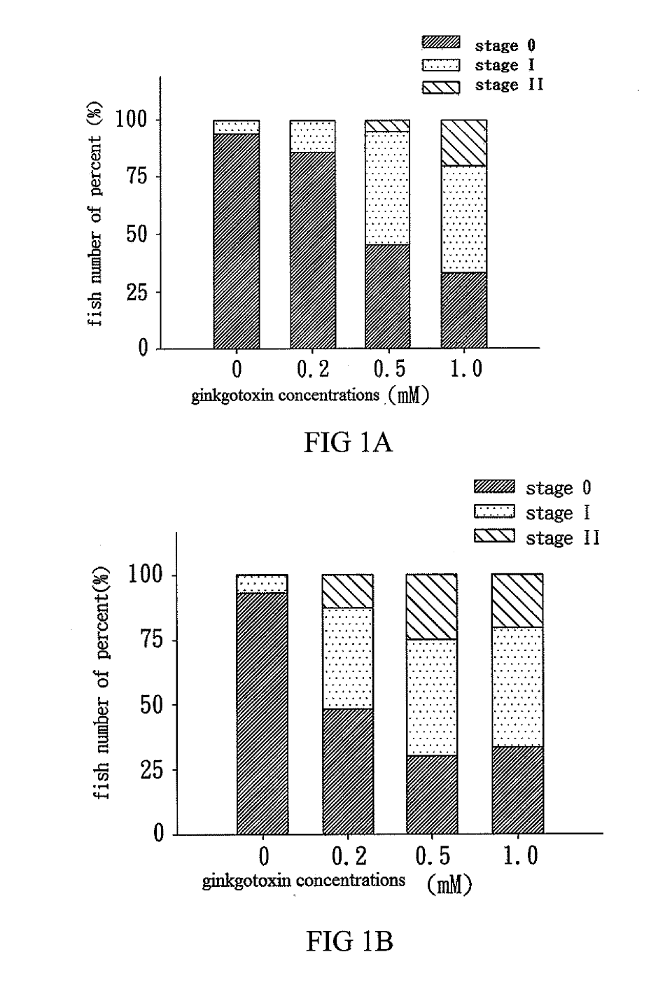

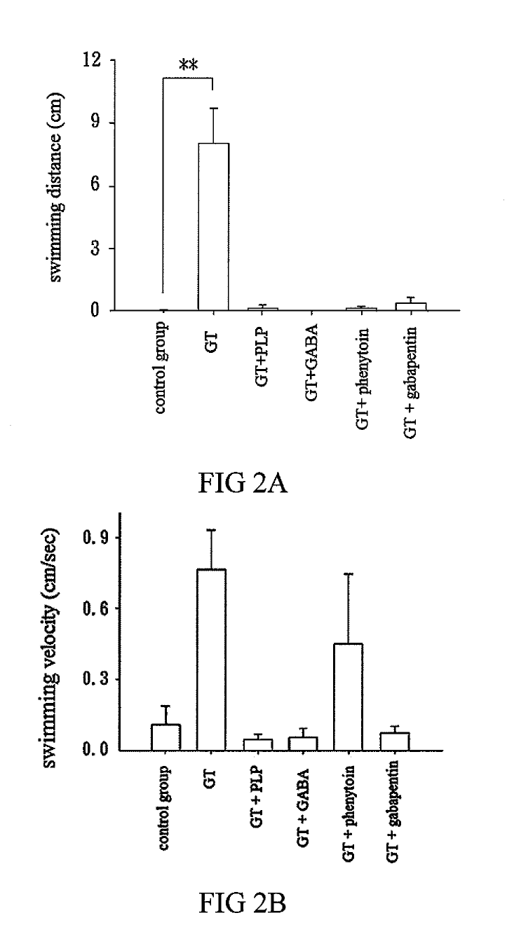

To induce seizures, the zebrafish larvae at 5 days post fertilization (5-dpf) are placed in fish water containing ginkgotoxin of various concentrations (0.2 mM to 1 mM) and ecorded and analyzed 2 hours later. The results are shown in FIGS. 1A and 1B, wherein stage 0 represents no or low influence, stage I represents obvious influence, and stage II represents twitch or convulsion.

As shown in FIGS. 1A and 1B, the zebrafish, whose activity is almost not influenced, is incubated in fish water without ginkgotoxin. However, the zebrafish vitality is adversely influenced to a more serious degree with increasing concentrations of ginkgotoxin, and the zebrafish seizure model can be established steadily at a concentration of 0.5 mM. Further, as shown in FIG. 1B, the zebrafish seizure model can be established with a lower dose (0.2 mM) after incubating for 2 hours.

Besides, the effects of ginkgotoxin phosphate are also similar to those of ginkgotoxin (results no shown).

As the result shown above, ginkgotoxin and phosphate thereof are used indeed in the zebrafish seizure model. Hereafter, the following experiments only use ginkgotoxin and the doses of 0.5 mM.

### Example 2 Screening for Drugs Using the Zebrafish Seizure Model

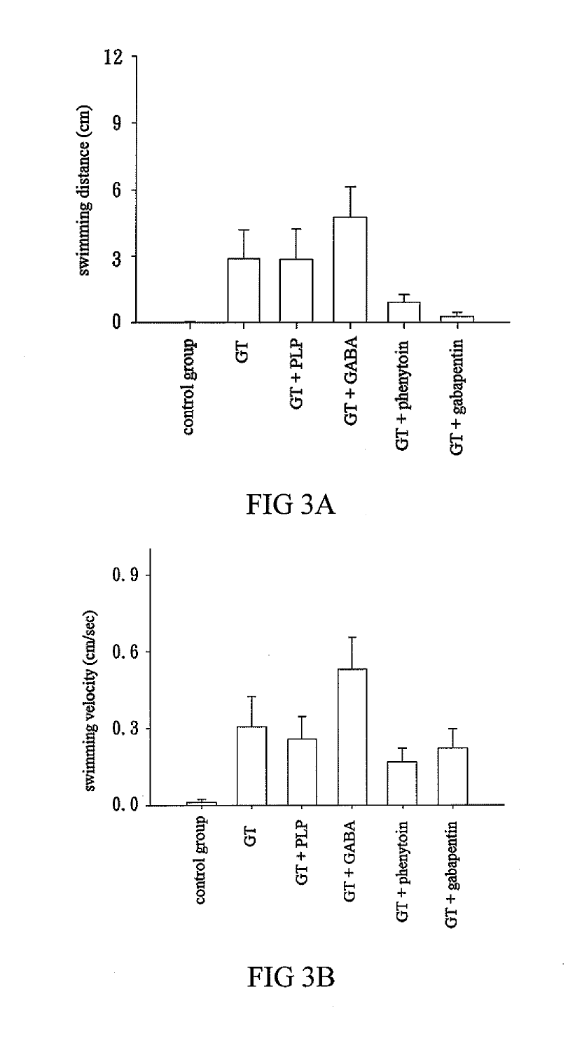

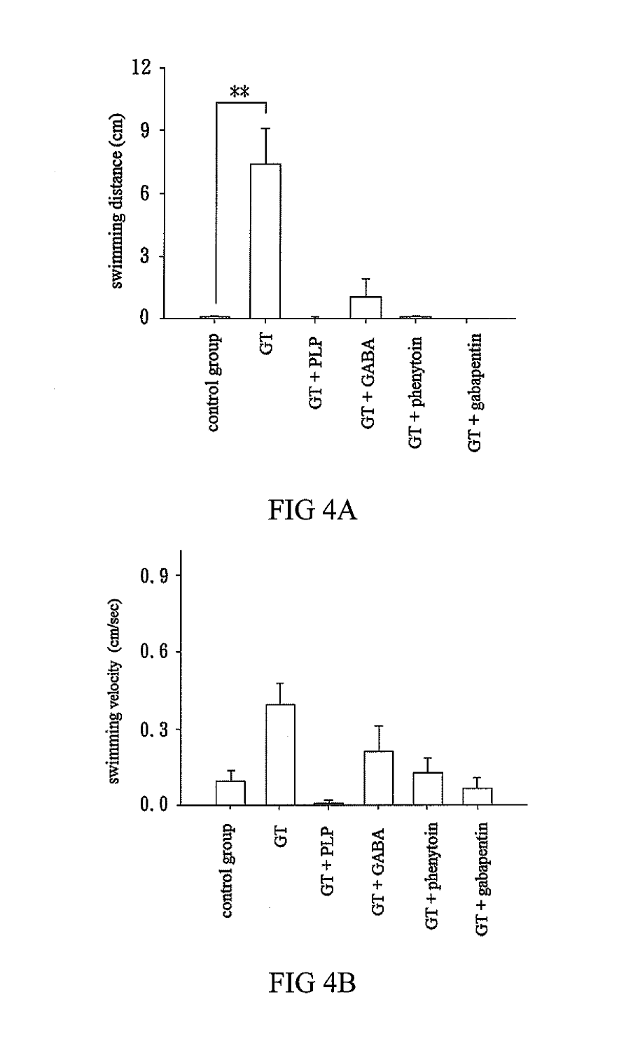

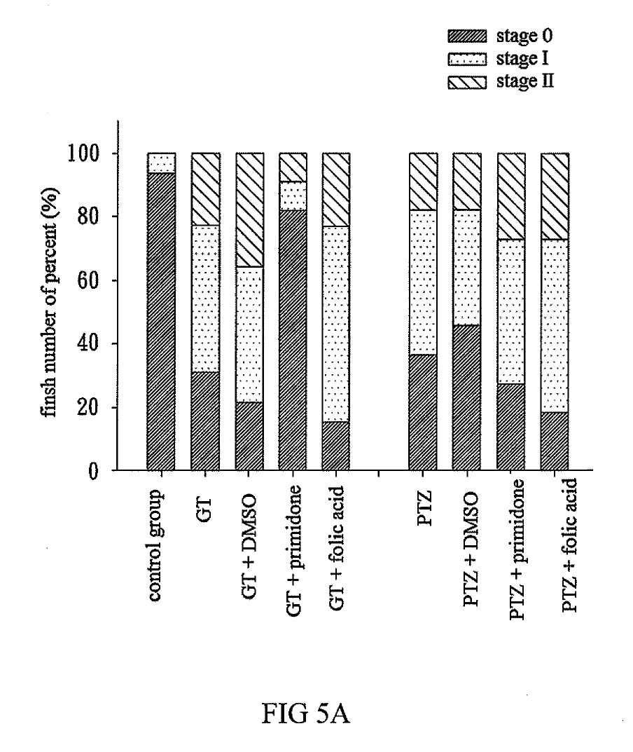

Herein, GABA, PLP, gabapentin and phenyloin are used to perform experiments. The zebrafish embryos are incubated in fish water without ginkgotoxin represented as a “control group”; the zebrafish embryos are treated with ginkgotoxin represented as “GT”; the zebrafish embryos are treated simultaneously with ginkgotoxin and PLP of 0.5 mM represented as “GT+PLP”; the zebrafish embryos are treated simultaneously with ginkgotoxin and GABA of 0.5 mM represented as “GT+GABA”; the zebrafish embryos are treated simultaneously with ginkgotoxin and gabapentin of 1 mM represented as “GT+gabapentin”; the zebrafish embryos are treated simultaneously with ginkgotoxin and phenyloin of 1 mM represented as “GT+phenyloin”. The results are shown in FIGS. 2A to 4B, wherein, FIG. 2A and FIG. 2B are results showing that zebrafish at 3 days post fertilization of the example are treated for 2 hours; FIG. 3A and FIG. 3B are results showing that zebrafish at 5 days post fertilization of the example are treated for 2 hours; FIG. 4A and FIG. 4B are results that zebrafish at 6 hours post fertilization of the example were treated for 3 days. These indicate aggrevated seizure and convulsion when the swimming distance is longer and swimming velocity is faster transitorily.

As shown in FIGS. 2A to 4B, the zebrafish (GT) treated with ginkgotoxin will all appear with states of seizure or convulsion, irrespective of the number of days post fertilization of zebrafish. Particularly, the zebrafish at 3 days post fertilization and zebrafish at six hours post fertilization exhibit the most obvious seizure and convulsion conditions, shown in FIGS. 2A, 2B, 4A and 4B.

Further, as shown in FIGS. 2A to 4B, the conditions of seizure and convulsion are alleviated with a greater extent when PLP or GABA is added simultaneously with GT. More importantly, zebrafish at six hours post fertilization show the most obvious signs of rescuing effect caused by PLP or GABA, shown in FIGS. 4A to 4B. This result proves that the zebrafish seizure model can not only be used for screening for antiepileptic drugs, but can also be used for screening for drugs targeting vitamin B6 deficiency.

Further, as shown in FIGS. 2A to 4B, the conditions of seizure and convulsion are alleviated with greater extent when gabapentin and phenyloin are added simultaneously, regardless of the embryonic stage. Particularly, zebrafish at six hours post fertilization show the most obvious signs of rescuing effect caused by gabapentin and phenyloin, shown in FIGS. 4A and 4B. This result shows that the zebrafish seizure model established in the example is suitable for drug screening for antiepileptic drugs and anti-convulsion drugs.

The present example only discloses the results for the zebrafish induced with ginkgotoxin and PLP/GABA/antiepileptic drugs all by the same batch. Also, similar results are observed when the embryos are treated with ginkgotoxin for two hours before PLP, GABA or antiepileptic drug is added and incubated for another three hours before data collection (Results not shown).

### Example 3 Comparing to the Present Zebrafish Seizure Model Induced With PTZ

Current experimental data show the seizure model induced by PTZ does not respond to the antiepileptic drug primidone. Here, a comparison between the zebrafish seizure model induced by PTZ and one induced by ginkgotoxin is drawn. Wherein, the zebrafish embryos incubated in fish water without ginkgotoxin represented as a “control group”; the zebrafish embryos only treated with ginkgotoxin represented as “GT”; the zebrafish embryos treated only with PTZ of 0.5 mM represented as “PTZ”; the zebrafish embryos treated simultaneously with ginkgotoxin and DMSO represented as “GT+DMSO”; the zebrafish embryos treated simultaneously with PTZ and DMSO represented as “PTZ+DMSO”; the zebrafish embryos treated simultaneously with ginkgotoxin and gabapentin of 2 mM represented as “GT+gabapentin”; the zebrafish embryos treated simultaneously with PTZ and phenyloin of 2 mM represented as “PTZ+phenyloin”; the zebrafish embryos treated simultaneously with GT and folic acid of 1 mM represented as “PTZ+phenyloin”; the zebrafish embryos treated simultaneously with PTZ and folic acid of 1 mM is indicated as “PTZ+folic acid”. Herein, folic acid serves as a negative control group.

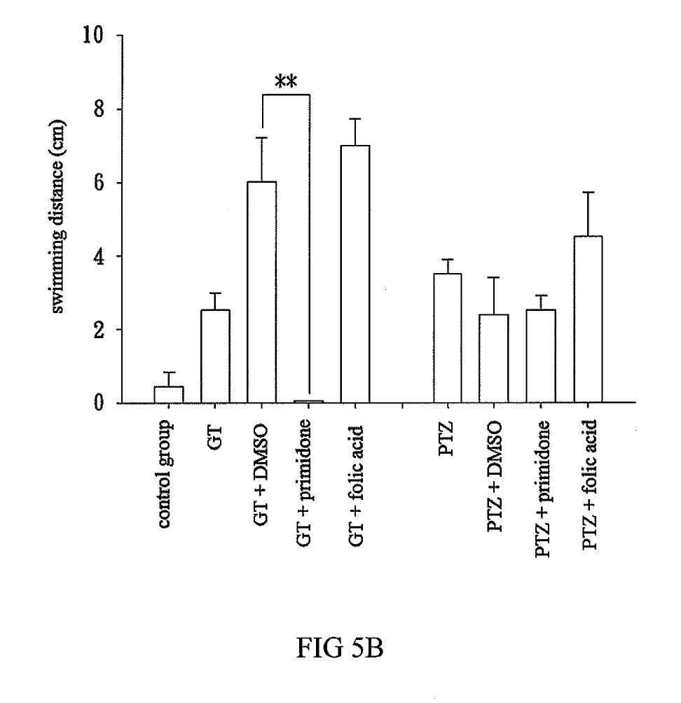

The results are shown in FIGS. 5A and 5B, wherein stage 0 represents no or low influence, stage I represents obvious influence, and stage II represents twitch or convulsion. This result shows that the zebrafish seizure model induced by ginkgotoxin enable to screen out primidone served as antiepileptic drugs or anti-convulsion drugs, which could not be accomplished by the zebrafish seizure model induced by PTZ.

Therefore, the present invention provides an alternative for zebrafish seizure model besides the present seizure model using PTZ. The zebrafish seizure model induced with ginkgotoxin has quite high reactivity and sensitivity in response to the antiepileptic drugs. Therefore, ginkgotoxin-induced zebrafish seizure model can serve as a screening platform for screening for antiepileptic drugs or anti-convulsion drugs.

The above embodiments are for the purpose of better description and are of exemplary nature only, the scope of right asserted by the current invention is based on the scope of claims in this application, and are not intended to be restricted by the above embodiments.

What is claimed is:

1. A method for establishing a zebrafish seizure model, comprising the following steps:
placing a zebrafish in a medium containing an inducing compound represented by the following formula (I) to induce seizure-like symptom in the zebrafish: 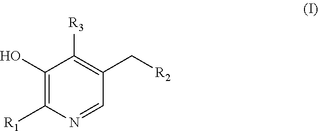
wherein, R₁ is an alkyl group having 1 to 6 carbon atoms, R₂ is a hydrogen atom, a hydroxyl group, or an alkyl group having 1 to 6 carbon atoms, and R₃ is an alkyl group having 1 to 6 carbon atoms, an alkoxy group having 1 to 6 carbon atoms, or an ether group having 1 to 6 carbon atoms.

2. The establishing method as claimed in claim 1, wherein the zebrafish is a zebrafish embryo.

3. The establishing method as claimed in claim 1, wherein R₁ is a methyl or ethyl, R₂ is a hydroxyl group, methyl or ethyl, and R₃ is an ether group having 1 to 6 carbon atoms.

4. The establishing method as claimed in claim 1, wherein R₃ is —C₁₋₃—O—C₁₋₃.

5. The establishing method as claimed in claim 1, wherein the induced compound is represented by the following formula (II):

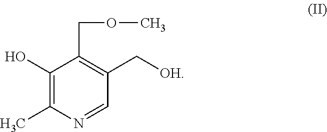

6. The establishing method as claimed in claim 5, wherein the medium is water.

7. The establishing method as claimed in claim 1, wherein a concentration of the inducing compound in the medium is 0.05 to 1.5 mM.

8. A zebrafish seizure model, the zebrafish seizure model is established by the following steps:
placing a zebrafish in a medium containing an inducing compound represented by the following formula (I) to induce seizure-like symptom in the zebrafish: 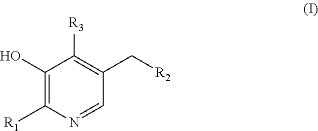
wherein, R₁ is an alkyl group having 1 to 6 carbon atoms, R₂ is a hydrogen atom, a hydroxyl group, or an alkyl group having 1 to 6 carbon atoms, and R₃ is a alkyl group having 1 to 6 carbon atoms, an alkoxy group having 1 to 6 carbon atoms, or an ether group having 1 to 6 carbon atoms.

9. The zebrafish seizure model as claimed in claim 8, wherein the zebrafish is a zebrafish embryo.

10. The zebrafish seizure model as claimed in claim 8, wherein R₁ is a methyl or ethyl, R₂ is a hydroxyl group, methyl or ethyl, R₃ is an ether group having 1 to 6 carbon atoms.

11. The zebrafish seizure model as claimed in claim 8, wherein R₃ is —C₁₋₃—O—C₁₋₃.

12. The zebrafish seizure model as claimed in claim 8, wherein the inducing compound is represented by the following formula (II):

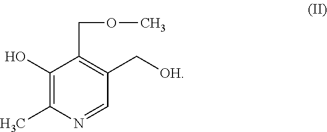

13. The zebrafish seizure model as claimed in claim 12, wherein the medium is a water.

14. The zebrafish seizure model as claimed in claim 8, wherein a concentration of the induced compound in the medium is 0.05 to 1.5 mM.

15. A method for screening for an antiepileptic drug, comprising the following the steps:
(A) providing a zebrafish seizure model, wherein the zebrafish seizure model is established by the following steps:
placing a zebrafish in a medium containing an inducing compound represented by the following formula (I) to induce seizure-like symptom in the zebrafish: 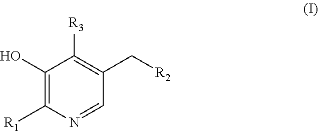
wherein, R₁ is an alkyl group having 1 to 6 carbon atoms, R₂ is a hydrogen atom, a hydroxyl group or an alkyl group having 1 to 6 carbon atoms, and R₃ is an alkyl group having 1 to 6 carbon atoms, an alkoxy group having 1 to 6 carbon atoms, or an ether group having 1 to 6 carbon atoms; and
(B) adding a drug candidate to the medium and observing the convulsion state of the zebrafish, wherein the drug candidate has antiepileptic effects when the convulsions state of the zebrafish is eased.

16. The screening method as claimed in claim 15, wherein the zebrafish is a zebrafish embryo.

17. The screening method as claimed in claim 15, wherein R₁ is a methyl or ethyl, R₂ is a hydroxyl group, methyl or ethyl, R₃ is an ether group having 1 to 6 carbon atoms.

18. The screening method as claimed in claim 15, wherein R₃ is —C₁₋₃—O—C₁₋₃.

19. The screening method as claimed in claim 15, wherein the induced compound is represented by the following formula (II):

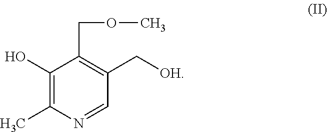

20. The screening method as claimed in claim 19, wherein the medium is a water.

21. The screening method as claimed in claim 15, wherein a concentration of the induced compound in the medium is 0.05 to 1.5 mM.
